(19) United States
(12) Patent Application Publication
(10) Pub. No.: US 2016/0193254 A1
(43) Pub. Date: Jul. 7, 2016

(54) METHODS AND MATERIALS FOR TREATING ARTHRITIS

(71) Applicant: Gary M. Petrucci, Long Lake, MN (US)

(72) Inventor: Gary M. Petrucci, Long Lake, MN (US)

(21) Appl. No.: 14/984,757

(22) Filed: Dec. 30, 2015

Related U.S. Application Data
(60) Provisional application No. 62/099,888, filed on Jan. 5, 2015.
(60) Provisional application No. 62/214,046, filed on Sep. 3, 2015.

(51) Int. Cl. A61K 35/50; A61K 35/28; A61K 9/00

(52) U.S. Cl. CPC A61K 35/50; A61K 9/0019; A61K 35/28

(57) ABSTRACT

This document provides methods and materials for treating arthritis. For example, compositions including an amnion tissue preparation and/or a stem cell preparation as well as methods for using such compositions to treat arthritis are provided.

### CLAIM OF PRIORITY

This application claims priority to U.S. Provisional Application Ser. No. 62/099,888, filed on Jan. 5, 2015 and U.S. Provisional Application Ser. No. 62/214,046 filed on Sep. 3, 2015, the entire contents of which are hereby incorporated by reference.

### BACKGROUND

1. Technical Field

This document relates to methods and materials for treating arthritis. For example, this document provides methods and materials for using compositions (e.g., injectable formulations) that include an amnion tissue preparation and/or a stem cell preparation to treat arthritis.

2. Background Information

Arthritis is a joint disorder where one or more joints are inflamed. The pain from the joint inflammation of arthritis can be localized to the particular inflamed joint and can be constant.

### SUMMARY

This document provides compositions that include an amnion tissue preparation and/or a stem cell preparation. Such compositions can be formulated for injection and used to treat arthritis (e.g., osteoarthritis, rheumatoid arthritis, psoriatic arthritis). This document also provides methods for using an amnion tissue preparation, a stem cell preparation, or both in combination to treat arthritis (e.g., osteoarthritis, rheumatoid arthritis, psoriatic arthritis).

In general, one aspect of this document features a method of treating arthritis in a mammal. The method comprises, or consists essentially of, administering, to the mammal, an amnion tissue preparation lacking viable cells (e.g., a dried amnion tissue preparation lacking viable cells) and a stem cell preparation having viable cells. The mammal can be a human. The arthritis can be osteoarthritis. The amnion tissue preparation can be administered by injection into a joint region of the mammal. The stem cell preparation can be administered by injection into a joint region of the mammal. The method can further comprise monitoring the arthritis of the mammal. The amnion tissue preparation can comprise an amnion tissue preparation prepared from about 1 mg to about 10 g of amnion tissue per kg of body weight of the mammal. In some cases, from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 mg, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation can be administered. The stem cell preparation can comprise from about 0.1 million to about 3 million (e.g., from about 0.3 million to about 3 million) stem cells per kg of body weight of the mammal. In some cases, the stem cell preparation can comprise from about 0.025 million to about 12 million (e.g., from about 2 million to about 6 million) stem cells per kg of body weight of the mammal. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans). The amnion tissue preparation can be a human amnion tissue preparation. The stem cell preparation can be a human mesenchymal stem cell preparation.

In another aspect, this document features a composition comprising a stem cell preparation having viable cells and an amnion tissue preparation lacking viable cells (e.g., a dried amnion tissue preparation lacking viable cells). The composition can comprise a therapeutic agent, a growth factor, or a pharmaceutical excipient. The composition can comprise from about 5 mg and about 5 g of the amnion tissue preparation. In some cases, the composition can comprise from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation. The composition can comprise from about 10 mg and about 1 g of the amnion tissue preparation. The stem cell preparation can comprise from about 10 million to about 100 million stem cells. In some cases, the stem cell preparation can comprise from about 0.025 million to about 12 million (e.g., from about 2 million to about 6 million) stem cells per kg of body weight of a mammal intended to receive the composition. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans).

In another aspect, this document features an injection device comprising a needle and a composition comprising an amnion tissue preparation lacking viable cells (e.g., a dried amnion tissue preparation lacking viable cells) and a stem cell preparation having viable cells. The composition can further comprise a therapeutic agent, a growth factor, or a pharmaceutical excipient. The composition can comprise from about 5 mg and about 5 g of the amnion tissue preparation. In some cases, the composition can comprise from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation. The composition can comprise from about 10 mg and about 1 g of the amnion tissue preparation. The stem cell preparation can comprise from about 10 million to about 100 million stem cells. In some cases, the stem cell preparation can comprise from about 0.025 million to about 12 million (e.g., from about 2 million to about 6 million) stem cells per kg of body weight of a mammal intended to receive the composition. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans). The device can be configured to administer a predetermined unit dose of the composition to a mammal.

In another aspect, this document features a method of treating arthritis in a mammal. The method comprises, or consists essentially of, administering, to the mammal, an amnion tissue preparation having viable cells and a stem cell preparation having viable cells. The mammal can be a human. The arthritis can be osteoarthritis. The amnion tissue preparation can be administered by injection into a joint region of the mammal. The stem cell preparation can be administered by injection into a joint region of the mammal. The method can further comprise monitoring the arthritis of the mammal. The amnion tissue preparation can comprise an amnion tissue preparation prepared from about 1 mg to about 10 g of amnion tissue per kg of body weight of the mammal. In some cases, from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation can be administered. The stem cell preparation can comprise from about 0.1 million to about 3 million stem (e.g., from about 0.3 million to about 3 million) cells per kg of body weight of the mammal. In some cases, the stem cell preparation can comprise from about 0.025 million to about 12 million (e.g., from about 2 million to about 6 million) stem cells per kg of body weight of a mammal. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans). The amnion tissue preparation can be a human amnion tissue preparation. The stem cell preparation can be a human mesenchymal stem cell preparation.

In another aspect, this document features a composition comprising a stem cell preparation having viable cells and an amnion tissue preparation having viable cells. The composition can comprise a therapeutic agent, a growth factor, or a pharmaceutical excipient. The composition can comprise from about 5 mg and about 5 g of the amnion tissue preparation. In some cases, the composition can comprise from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation. The composition can comprise from about 10 mg and about 1 g of the amnion tissue preparation. The stem cell preparation can comprise from about 10 million to about 100 million stem cells. In some cases, the stem cell preparation can comprise from about 0.025 million to about 12 million (e.g., from about 2 million to about 6 million) stem cells per kg of body weight of a mammal intended to receive the composition. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans).

In another aspect, this document features an injection device comprising a needle and a composition comprising an amnion tissue preparation having viable cells and a stem cell preparation having viable cells. The composition can further comprise a therapeutic agent, a growth factor, or a pharmaceutical excipient. The composition can comprise from about 5 mg and about 5 g of the amnion tissue preparation. The composition can comprise from about 10 mg and about 1 g of the amnion tissue preparation. In some cases, the composition can comprise from about 0.01 mg to about 10 g (e.g., from about 0.01 mg to about 10 g, from about 0.1 mg to about 10 g, from about 1 mg to about 10 g, from about 1 mg to about 10 g, from about 5 mg to about 5 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 10 mg to about 1 g, from about 100 mg to about 1 g, or from about 1 g to about 5 g) of the amnion tissue preparation. The stem cell preparation can comprise from about 10 million to about 100 million stem cells. In some cases, the stem cell preparation can comprise from about 0.025 million to about 12 million (e.g., from about 2 million to about 6 million) stem cells per kg of body weight of a mammal intended to receive the composition. For example, when administering a stem cell preparation to a rat weighing about 250 g, the stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans). The device can be configured to administer a predetermined unit dose of the composition to a mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims. The word “comprising” in the claims may be replaced by “consisting essentially of” or with “consisting of,” according to standard practice in patent law.

### DETAILED DESCRIPTION

In general, this document provides methods and materials for treating arthritis (e.g., osteoarthritis, rheumatoid arthritis, psoriatic arthritis) using compositions that include an amnion tissue preparation (e.g., human amnion tissue preparation) and/or a stem cell preparation (e.g., a human stem cell preparation).

The term “amnion tissue preparation” as used herein refers to a preparation of amnion tissue or amnion material. In some cases, an amnion tissue preparation can be a liquid preparation (e.g., solution or suspension) that is prepared from a dried amnion tissue preparation. The term “dried amnion tissue preparation” as used herein refers to a preparation of amnion tissue or amnion material that is dried to have a water content that is less than about 8 percent (e.g., less than about 7 percent, less than about 6 percent, less than about 5 percent, less than about 4 percent, less than about 3 percent, less than about 2 percent, or less than about 1 percent).

The term “stem cell preparation” as used herein refers to a preparation of stem cells or stem cell material. In some cases, a stem cell preparation can be a liquid preparation (e.g., solution or suspension) that is prepared from a dried stem cell preparation. The term “dried stem cell preparation” as used herein refers to a preparation of stem cells or stem cell material that is dried to have a water content that is less than about 8 percent (e.g., less than about 7 percent, less than about 6 percent, less than about 5 percent, less than about 4 percent, less than about 3 percent, less than about 2 percent, or less than about 1 percent). In some cases, a dried amnion tissue preparation or a dried stem cell preparation can have a water content that is between about 0.1 percent and about 8 percent (e.g., between about 0.5 percent and about 8 percent, between about 1 percent and about 8 percent, between about 0.1 percent and about 5 percent, between about 0.1 percent and about 4 percent, between about 0.1 percent and about 3 percent, between about 0.5 percent and about 5 percent, or between about 1 percent and about 4 percent). An amnion tissue preparation or stem cell preparation can be dried using any appropriate technique such as micronization, vacuum drying, spray drying, freeze drying, or combinations thereof. In some cases, an amnion tissue preparation or stem cell preparation can be dried as described elsewhere (e.g., U.S. Pat. No. 5,656,498).

An amnion tissue preparation or a dried amnion tissue preparation can contain viable cells, non-viable cells, or a combination thereof. For example, an amnion tissue preparation or a dried amnion tissue preparation can be a preparation of amnion tissue or amnion material having viable cells. In some cases, an amnion tissue preparation can be a solution or suspension of amnion tissue or amnion material having viable cells.

In some cases, an amnion tissue preparation or a dried amnion tissue preparation can be a preparation of amnion tissue or amnion material where all the cells were removed, killed, or lysed such that the amnion tissue preparation or the dried amnion tissue preparation lacks viable cells. In some cases, an amnion tissue preparation or a dried amnion tissue preparation can be a preparation of amnion tissue or amnion material that was exposed to one or more physical and/or chemical treatments that killed, fixed, or lysed the cells of the amnion tissue or amnion material such that the amnion tissue preparation or the dried amnion tissue preparation lacks viable cells. For example, temperature (e.g., rapid freezing or rapid freezing-thawing), force and pressure, and/or electrical disruption can be used to kill or lyse cells within amnion tissue or amnion material to produce an amnion tissue preparation or a dried amnion tissue preparation that lacks viable cells.

In some cases, amnion tissue or amnion material can be obtained and then treated in a manner designed to lyse all the cells within the amnion tissue or amnion material. In these cases, the resulting material (e.g., matrix material and cellular remnants from lysed cells) can be used as an amnion tissue preparation that lacks viable cells or dried to form a dried amnion tissue preparation that lacks viable cells.

In some cases, an amnion tissue preparation or a dried amnion tissue preparation can be prepared from human amnion tissue. For example, human amnion tissue can be harvested, processed to maintain cell viability with or without removing blood, and used as an amnion tissue preparation or dried to form a dried amnion tissue preparation.

In some cases, human amnion tissue can be processed to remove blood prior to being used as an amnion tissue preparation or prior to being dried to form a dried amnion tissue preparation. In some cases, human amnion tissue can be processed without removing cells or blood prior to forming an amnion tissue preparation or a dried amnion tissue preparation.

An example of an amnion tissue preparation includes, without limitation, a human amnion tissue preparation that includes viable cells. In some cases, an amnion tissue preparation can be obtained from MiMedX® or a tissue bank (e.g., a human tissue bank).

A stem cell preparation or a dried stem cell preparation can contain viable stem cells, non-viable stem cells, or a combination thereof. For example, a stem cell preparation or a dried stem cell preparation can be a preparation of viable stem cells. In some cases, a stem cell preparation can be a solution or suspension of viable stem cells.

In some cases, a stem cell preparation or a dried stem cell preparation can be a preparation of stem cell or stem cell material where all the stem cells were killed, fixed, or lysed such that the stem cell preparation lacks viable stem cells or the dried stem cell preparation lacks viable stem cells. In some cases, a stem cell preparation or a dried stem cell preparation can be a preparation of stem cells or stem cell material that was exposed to one or more physical and/or chemical treatments that killed, fixed, or lysed the stem cells such that the stem cell preparation lacks viable stem cells or the dried stem cell preparation lacks viable stem cells. For example, temperature (e.g., rapid freezing or rapid freezing-thawing), force and pressure, and/or electrical disruption can be used to kill or lyse stem cells to produce a stem cell preparation that lacks viable stem cells or a dried stem cell preparation that lacks viable stem cells.

In some cases, a stem cell culture can be obtained and then used as a stem cell preparation in a manner that maintains stem cell viability.

Examples of stem cell preparations include, without limitation, a lung stem cell preparation such as a lung epithelial progenitor cell preparation, a mesenchymal stem cell (MSC) preparation (e.g., a MSC preparation obtained from fat tissue or bone marrow), an umbilical cord blood stem cell preparation, an embryonic stem cell preparation, and a human induced pluripotent stem cell preparation.

In some cases, stem cell preparations and dried stem cell preparations are prepared from cultures of stem cells. For example, a culture containing from about 25 million to about 25 billion stem cells can be used to make a stem cell preparation or a dried stem cell preparation. In some cases, from about 0.1 million to about 3 million (e.g., from about 0.3 million to about 3 million, from about 0.5 million to about 3 million, from about 0.75 million to about 3 million, from about 1 million to about 3 million, from about 1.5 million to about 3 million, from about 0.3 million to about 2.5 million, from about 0.3 million to about 2.0 million, from about 0.3 million to about 1.5 million, from about 0.3 million to about 1.0 million, from about 0.5 million to about 2.5 million, from about 0.75 million to about 2.0 million, from about 0.8 million to about 1.5 million) stem cells per kg of body weight of a mammal (e.g., a human) to be treated can be used to make a stem cell preparation or a dried stem cell preparation for administration to that mammal. In some cases, a stem cell preparation can include from about 0.025 million to about 12 million (e.g., from about 2 million to about 6 million) stem cells per kg of body weight of a mammal intended to receive the stem cell preparation. For example, when administering a stem cell preparation to a rat weighing about 250 g, a stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans). In some cases, a stem cell preparation and an amnion tissue preparation can be formulated into a single solution or suspension for administration to a mammal. For example, a dried amnion tissue preparation can be reconstituted into a solution and a stem cell preparation can be added to that solution to form a single solution or suspension having both a stem cell preparation and an amnion tissue preparation. In some cases, a stem cell preparation can be obtained commercially from a variety of suppliers such as Stemedica Cell Technologies, Inc.

In some cases, a dried stem cell preparation can be prepared by washing a culture of stem cells in saline (e.g., phosphate buffered saline) to remove culture medium, evaporating to remove wash medium, adding a solution (e.g., saline, water, or a water and sugar solution) to the resulting stem cell preparation, and repeating the evaporation step. After the second evaporation step, the stem cell preparation can be formulated into a powder that can be used as a dried stem cell preparation. The powder can be exposed to a liquid (e.g., saline) to create a solution for administration to a mammal (e.g., a human).

A dried amnion tissue preparation and/or a dried stem cell preparation can have any appropriate particle size. For example, a dried amnion tissue preparation and/or a dried stem cell preparation can have a particle size ranging from about 0.1 μm to about 25 μm (e.g., from about 0.5 μm to about 25 μm, from about 0.75 μm to about 25 μm, from about 1 μm to about 25 μm, from about 0.1 μm to about 15 μm, from about 0.1 μm to about 10 μm, from about 0.1 μm to about 7.5 μm, from about 0.1 μm to about 5 μm, from about 0.75 μm to about 7.5 μm, or from about 1 μm to about 5 μm).

Typically, a composition described herein (e.g., a composition containing an amnion tissue preparation lacking viable cells and a stem cell preparation having viable stem cells or a composition containing an amnion tissue preparation having viable cells, a stem cell preparation having viable stem cells, or both an amnion tissue preparation having viable cells and a stem cell preparation having viable stem cells) is administered via injection or infusion. For example, a composition containing an amnion tissue preparation lacking viable cells (e.g., a dried amnion tissue preparation) and a stem cell preparation having viable stem cells can be injected into an inflamed joint region of a mammal having arthritis. In some cases, a composition containing an amnion tissue preparation having viable cells and a stem cell preparation having viable stem cells can be injected into an inflamed joint region of a mammal having arthritis. In some cases, a composition described herein (e.g., a composition containing an amnion tissue preparation lacking viable cells and a stem cell preparation having viable stem cells or a composition containing an amnion tissue preparation having viable cells, a stem cell preparation having viable stem cells, or both an amnion tissue preparation having viable cells and a stem cell preparation having viable stem cells) is administered during an arthrotomy procedure.

In some cases, a composition that includes an amnion tissue preparation (e.g., an amnion tissue preparation having viable cells or an amnion tissue preparation lacking viable cells) and/or a stem cell preparation (e.g., a stem cell preparation having viable stem cells) also can include one or more therapeutic agents, one or more anti-inflammatory agents (e.g., non-steroidal anti-inflammatory drugs, dexamethasone or other type of glucocorticoid steroids), one or more growth factors (e.g., platelet derived growth factor PDGF, epithelial growth factor (EGF), fibroblast growth factor-2 (FGF2), or stem cell factor (SCF)), and/or one or more antimicrobial agents (e.g., antibiotics such as kanamycin, neomycin, streptomycin, or gentamicin or an antifungal agent).

As described herein, arthritis conditions can be treated by administering (e.g., via injection such as an intravenous injection or an injection into a joint region) an effective amount of a composition that includes an amnion tissue preparation described herein (e.g., an amnion tissue preparation having viable cells or an amnion tissue preparation lacking viable cells) and/or a stem cell preparation described herein (e.g., a stem cell preparation having viable stem cells). Effective amounts of compositions described herein can be determined by a physician, taking into account various factors such as overall health status, body weight, sex, diet, time and route of administration, other medications, and any other relevant clinical factors. As used herein, an “effective amount” or “therapeutically effective amount” of a composition provided herein is the amount that is sufficient to provide a beneficial effect to the subject to which the composition or preparations are delivered. The effective amount can be the amount effective to achieve an improved reduction in joint inflammation or joint pain, a more rapid recovery, an improvement in the quality of life, or an improvement or elimination of one or more symptoms associated with a subject's arthritis.

In some embodiments, the methods include delivering, to the subject, an amnion tissue preparation (e.g., an amnion tissue preparation having viable cells or an amnion tissue preparation lacking viable cells) made with from about 0.01 mg to about 10 g (e.g., from about 0.01 mg to about 10 g, from about 0.1 mg to about 10 g, from about 1 mg to about 10 g, from about 10 mg to about 10 g, from about 100 mg to about 10 g, from about 1 g to about 10 g, from about 0.01 mg to about 5 g, from about 0.01 mg to about 1 g, from about 0.01 mg to about 100 mg, from about 10 mg to about 5 g, from about 100 mg to about 1 g, or from about 1 g to about 5 g) of amnion tissue per kg body weight of the subject being treated.

In some embodiments, the methods include delivering, to the subject, a stem cell preparation (e.g., a stem cell preparation having viable stem cells) made from about 0.1 million to about 3 million (e.g., from about 0.3 million to about 3 million, from about 0.5 million to about 3 million, from about 0.75 million to about 3 million, from about 1 million to about 3 million, from about 1.5 million to about 3 million, from about 0.3 million to about 2.5 million, from about 0.3 million to about 2.0 million, from about 0.3 million to about 1.5 million, from about 0.3 million to about 1.0 million, from about 0.5 million to about 2.5 million, from about 0.75 million to about 2.0 million, from about 0.8 million to about 1.5 million) stem cells per kg body weight of the subject being treated. In some cases, a stem cell preparation can include from about 0.025 million to about 12 million (e.g., from about 2 million to about 6 million) stem cells per kg of body weight of a mammal intended to receive the stem cell preparation. For example, when administering a stem cell preparation to a rat weighing about 250 g, a stem cell preparation can include between about 0.75 million and 1.25 million stem cells. The volume of such an administration can be from about 45 μL to about 65 μL (e.g., about 50-60 μL). These amounts and volumes can be increased appropriately for larger mammals (e.g., cats, dogs, horses, or humans).

In some embodiments, a composition containing an amnion tissue preparation (e.g., an amnion tissue preparation having viable cells or an amnion tissue preparation lacking viable cells) and/or a stem cell preparation (e.g., a stem cell preparation having viable stem cells) is delivered to the subject (e.g., by injection) only once. In some embodiments, multiple (e.g., two, three, four, five, six, seven, eight, nine, 10, 11, 12, 13, 14, 15, or 20 or more) administration can be used. For example, multiple deliveries of a composition containing an amnion tissue preparation (e.g., an amnion tissue preparation having viable cells or an amnion tissue preparation lacking viable cells) and/or a stem cell preparation (e.g., a stem cell preparation having viable stem cells) can be made over the course of several (e.g., two, three, four, five, six, seven, eight, nine, 10, 14, 21, 28, or 31 or more) consecutive days (e.g., one delivery each day for seven days or one delivery every other day for seven days). In some cases, a composition containing an amnion tissue preparation (e.g., an amnion tissue preparation having viable cells or an amnion tissue preparation lacking viable cells) and/or a stem cell preparation (e.g., a stem cell preparation having viable stem cells) can be delivered from about once a week to about once per year (e.g., once every month or once every other month). In some cases, a composition containing an amnion tissue preparation (e.g., an amnion tissue preparation having viable cells or an amnion tissue preparation lacking viable cells) and/or a stem cell preparation (e.g., a stem cell preparation having viable stem cells) can be delivered to a subject for several months (e.g., one delivery per month for six months, or one delivery per week for two months).

A composition containing an amnion tissue preparation (e.g., an amnion tissue preparation having viable cells or an amnion tissue preparation lacking viable cells) and/or a stem cell preparation (e.g., a stem cell preparation having viable stem cells) can be delivered to a subject at various time points after diagnosis with arthritis. For example, a composition containing an amnion tissue preparation (e.g., an amnion tissue preparation having viable cells or an amnion tissue preparation lacking viable cells) and/or a stem cell preparation (e.g., a stem cell preparation having viable stem cells) can be delivered immediately following diagnosis of arthritis. In some cases, a composition containing an amnion tissue preparation (e.g., an amnion tissue preparation having viable cells or an amnion tissue preparation lacking viable cells) and/or a stem cell preparation (e.g., a stem cell preparation having viable stem cells) can be delivered to a subject less than 10 (e.g., 9, 8, 7, 6, 5, 4, 3, 2, or 1) days after diagnosis with arthritis.

The subject can be any mammal, e.g., a human (e.g., a human patient) or a non-human primate (e.g., chimpanzee, baboon, or monkey), a mouse, a rat, a rabbit, a guinea pig, a gerbil, a hamster, a horse, a camel, a type of livestock (e.g., cow, pig, sheep, or goat), a mammalian zoo animal (e.g., a lion, a tiger, or a leopard), a dog, or a cat.

A composition containing an amnion tissue preparation (e.g., an amnion tissue preparation having viable cells or an amnion tissue preparation lacking viable cells) and/or a stem cell preparation (e.g., a stem cell preparation having viable stem cells) can be administered to a subject as a combination therapy with another treatment used to treat arthritis. For example, a combination therapy can include administering to the subject (e.g., a human patient) one or more additional agents that provide a therapeutic benefit to the subject who has, or is at risk of developing arthritis. In some cases, the composition and the one or more additional agents can be administered at the same time. In some cases, the composition can be administered first, and the one or more additional agents administered second, or vice versa.

The efficacy of a given treatment in treating arthritis can be defined as an improvement of one or more symptoms of the arthritis by at least 5% (e.g., at least 10%, at least 15%, at least 20%, at least 25%, at least 30%, at least 40%, at least 50%, at least 55%, at least 60%, at least 65% or more). In some cases, efficacy of a treatment with a composition containing an amnion tissue preparation (e.g., an amnion tissue preparation having viable cells or an amnion tissue preparation lacking viable cells) and/or a stem cell preparation (e.g., a stem cell preparation having viable stem cells) can be determined from the stabilization of one or more symptoms associated with arthritis (i.e., the treatments curtail the worsening of one or more symptoms of arthritis).

In some cases, the methods described herein can include monitoring arthritis in the subject to, for example, determine if the arthritis is improving with treatment. Any appropriate method can be used to monitor arthritis. For example, joint pain can be monitored.

A composition containing an amnion tissue preparation (e.g., an amnion tissue preparation having viable cells or an amnion tissue preparation lacking viable cells) and/or a stem cell preparation (e.g., a stem cell preparation having viable stem cells) can be combined with packaging material and sold as a kit. The packaging material included in a kit typically contains instructions or a label describing how the composition can be administered via, for example, injection such as an intravenous injection or an injection into a joint region. A kit also can include a unit dose injector. The term “unit dose injector” refers to an injection device that delivers a single dose of a composition containing an amnion tissue preparation (e.g., an amnion tissue preparation having viable cells or an amnion tissue preparation lacking viable cells) and/or a stem cell preparation (e.g., a stem cell preparation having viable stem cells) by injection into a user. Typically, a unit dose injector contains a single container that holds or contains an injectable formulation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating arthritis in a mammal, said method comprising administering, to said mammal, an amnion tissue preparation lacking viable cells and a stem cell preparation having viable cells.

2. The method of claim 1, wherein said mammal is a human.

3. The method of claim 1, wherein said arthritis is osteoarthritis.

4. The method of claim 1, wherein said amnion tissue preparation is administered by injection into a joint region of said mammal.

5. The method of claim 1, wherein said stem cell preparation is administered by injection into a joint region of said mammal.

6. The method of claim 1, wherein said method further comprises monitoring said arthritis of said mammal.

7. The method of claim 1, wherein said amnion tissue preparation comprises an amnion tissue preparation prepared from about 1 mg to about 10 g of amnion tissue per kg of body weight of said mammal.

8. The method of claim 1, wherein said stem cell preparation comprises from about 0.3 million to about 3 million stem cells per kg of body weight of said mammal.

9. The method of claim 1, wherein said amnion tissue preparation is a human amnion tissue preparation.

10. The method of claim 1, wherein said stem cell preparation is a human mesenchymal stem cell preparation.

11. A composition comprising a stem cell preparation having viable cells and an amnion tissue preparation lacking viable cells.

12. The composition of claim 11, further comprising a therapeutic agent, a growth factor, or a pharmaceutical excipient.

13. The composition of claim 11, wherein said composition comprises from about 5 mg and about 5 g of said amnion tissue preparation.

14. The composition of claim 11, wherein said composition comprises from about 10 mg and about 1 g of said amnion tissue preparation.

15. The composition of claim 11, wherein said stem cell preparation comprises from about 10 million to about 100 million stem cells.

16. An injection device comprising a needle and a composition comprising an amnion tissue preparation lacking viable cells and a stem cell preparation having viable cells.

17. The device of claim 16, wherein said composition further comprises a therapeutic agent, a growth factor, or a pharmaceutical excipient.

18. The device of claim 16, wherein said composition comprises from about 5 mg and about 5 g of said amnion tissue preparation.

19. The device of claim 16, wherein said composition comprises from about 10 mg and about 1 g of said amnion tissue preparation.

20. The device of claim 16, wherein said stem cell preparation comprises from about 10 million to about 100 million stem cells.
